SEQUENCE LISTING (9 sequences)

SEQ ID NO: 1 - protein, 16 aa, Artificial Sequence
  Description of Artificial Sequence Synthetic amino acid sequence
DCECLGGGRISHQSQD

SEQ ID NO: 2 - protein, 33 aa, Artificial Sequence
  Description of Artificial Sequence Synthetic amino acid sequence
DCECLGGGRISHQSQDXKIHVYGYSMXYGXAQH

SEQ ID NO: 3 - protein, 44 aa, Artificial Sequence
  Description of Artificial Sequence Synthetic amino acid sequence
YHADIYDKVSGDMQKQGCDCECLGGGRISHQSQDKKIHVYGYSM

SEQ ID NO: 4 - protein, 125 aa, Artificial Sequence
  Description of Artificial Sequence Synthetic amino acid sequence
MAVADLALIPDVDIDSDGVFKYVLIRVHSAPRSGAPAAESKEIVRGYKWAEYHADIYDKVSGDMQKQGCDCECLGGGRISHQSQDKKIHVYGYSMAYGPAQHAISTEKIKAKYPDYEVTWANDGY

SEQ ID NO: 5 - protein, 12 aa, Artificial Sequence
  Description of Artificial Sequence Synthetic amino acid sequence
CLGGGRISHQDK

SEQ ID NO: 6 - DNA, 375 bp, Artificial Sequence
  Description of Artificial Sequence Synthetic nucleotide sequence
taccgccaccgcctggagcgagagtaaggactacacctgtagctgaggctgccgcagaagttcatacacgactaggctcaggtgagccgaggggcgaggccccgaggccgacgtctctcgttcctctagcacgcgccgatgttcacccgactcatggtacgcctgtagatgctgtttcacagcccgctgtacgtcttcgttccgacgctgacactcacagacccgccgcccgcgtagagggtggtctcagtcctgttcttctaagtgcacatgccgataaggtaccggataccaggacgggtcgtgcggtaaagttgactcttttagtttcggttcatggggctgatgctccagtggacccgattgctgccgatg

SEQ ID NO: 7 - DNA, 48 bp, Artificial Sequence
  Description of Artificial Sequence Synthetic nucleotide sequence
ctgacactcacagacccgccgcccgcgtagagggtggtctcagtcctg

SEQ ID NO: 8 - DNA, 99 bp, Artificial Sequence
  Description of Artificial Sequence Synthetic nucleotide sequence
ctgacactcacagacccgccgcccgcgtagagggtggtctcagtcctgttcttctaagtgcacatgccgataaggtaccggataccaggacgggtcgtg

SEQ ID NO: 9 - DNA, 132 bp, Artificial Sequence
  Description of Artificial Sequence Synthetic nucleotide sequence
atggtacgcctgtagatgctgtttcacagcccgctgtacgtcttcgttccgacgctgacactcacagacccgccgcccgcgtagagggtggtctcagtcctgttcttctaagtgcacatgccgataaggtac

(19) United States
(12) Patent Application Publication
(10) Pub. No.: US 2006/0153825 A1
(43) Pub. Date: Jul. 13, 2006

(54) Use of protein histidine phosphatase

(75) Inventors: Roland Kellner, Heppenheim (DE); Susanne Klumpp, Munster (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/471,100

(22) PCT Filed: Mar. 4, 2002
(86) PCT No.: PCT/EP02/02296

§ 371 Date: Sep. 8, 2003

(30) Foreign Application Priority Data
Mar. 8, 2001 (EP) 01105774.2

(51) Int. Cl. A61K 38/46; A61K 39/395; C12N 9/16; C12P 21/06

(52) U.S. Cl. 424/94.6; 424/146.1; 435/194; 435/69.1; 435/196; 435/320.1

(57) ABSTRACT

The invention relates to the use of polypeptides with protein histidine phosphatase activity derived from mammalians, antibodies directed against them and DNA or RNA sequences complementary to mRNA sequences encoding polypeptides with protein histidine phosphatase activity for the modulation of ATP-citrate lyase and treatment of correlated pathophysiologic functions.

The invention relates to the use of polypeptides with protein histidine phosphatase activity derived from mammalians, antibodies directed to these polypeptides and DNA or RNA sequences complementary to mRNA sequences encoding polypeptides with protein histidine phosphatase activity for the modulation of ATP-citrate lyase and the treatment of correlated pathophysiologic functions.

### BACKGROUND OF THE INVENTION

Post-translational modifications such as protein phosphorylation provide an important mechanism by which the functional activity of proteins can be controlled and, hence, biological processes regulated. Protein kinases and phosphatases are involved in the regulation of diverse cellular functions, including differentiation, growth control, tumor promotion, cell cycle and cell death. Phosphorylation/dephosphorylation of key enzymes of metabolic or anabolic pathways on specific residues has emerged as a central mechanism for the up- or down-regulation of such key enzymes.

ATP-citrate lyase (ACL; EC 4.1.3.8), the key enzyme for providing cytosolic acetyl-CoA, is a tetramer of four apparently identical subunits (Singh, M. et al. (1976) J. Biol. Chem., 251, 5242-5250) having the highest activity in liver, brain and kidney (Srere, P. A. (1959) J. Biol. Chem. 234, 2544-2547). ACL gene expression and protein content are increased at the transcriptional level by caloric intake and insulin, and are decreased by starvation and in diabetes mellitus (Towle, H. C. et al. (1997) Annu. Rev. Nutr. 17, 405-433; Rosiers, S. D. et al. (1995) J. Biol. Chem., 270, 10027-10033). The enzyme has three regulatory phosphorylation sites and in vivo phosphorylation of ACL at these sites changes in response to nutrients, the hormonal milieu and during differentiation (Benjamin, W. B. et al. (1994) Biochem. J., 300, 477-482).

ACL catalyzes the formation of acetyl-CoA and oxaloacetate in the cytosol from citrate and CoA with the hydrolysis of ATP to ADP and phosphate. This step is the major source of cytosolic acetyl-CoA which is used in the biosynthetic pathways of carbohydrates, fatty acids, cholesterol and acetyl choline.

The enzyme follows a mechanism with a phosphoenzyme intermediate (Plowman, K. M. et al., (1967) J. Biol. Chem. 242, 4239-4247; Wells, T. N. C. (1991) Eur. J. Biochem. 199, 163-168) resulting from phosphorylation of the enzyme at the catalytic site by the substrate ATP in the first step of the overall reaction. This phosphorylation site is at His 760 (Williams, S. P. et al. (1985) Biochem., 24, 5527-5531).

Recent findings suggest that ACL may also play an important role in gluconeogenesis, as it catalyzes the formation of a significant portion of cytosolic oxaloacetate, a major gluconeogenic precursor (Rosiers, S. D. et al. (1995) J. Biol. Chem., 270, 10027-10033). Furthermore, ACL activity changes, by regulating the cytosolic concentration of citrate, could modulate both glycolysis, by inhibition of phosphofructokinase (Comte, B. et al. (1997) J. Biol. Chem., 272, 26117-26124), and fatty acid biosynthesis, by allosteric activation of acetyl-CoA carboxylase (Reilly, D. I. et al. (1997) Prog. Lipid Res., 35, 371-385).

The reaction catalyzed by ACL is the key supply of acetyl-CoA for lipogenesis, and cholesterogenesis. Studies have demonstrated, that inhibition of this enzyme leads to a decrease in the synthesis of both cholesterol and fatty acids and an increase in low-density lipoprotein receptor activity suggesting a potential utility of an ACL inhibitor as hypolipidaemic drug (Berkout, T. A. et al (1990) Biochem. J., 272, 181-186), as a drug inducing weight loss (WO 97/18806) or as a drug for the treatment of obesity.

A further important pathway wherein ACL is involved is the synthesis of the neurotransmitter acetyl choline. Acetyl-CoA, converted from citrate by ACL is combined with choline through the action of choline acetyl transferase in cytosol. Because deficiency of acetyl choline is one characteristic of Alzheimer's disease and clinically improvement in symptoms can occur by treatment with acetyl choline esterase inhibitors (Bartus, R. T. et al. (1982) Science, 217, 408-414) ACL might play a important role in Alzheimer's disease and other types of dementia. In carcinoma of different organs a high level of expression of fatty acid synthase is observed (Kuhajda, F. P. et al. (1994) Proc. Natl. Acad. Sci. U.S.A., 91, 6379-6383; Rashid, A. et al. (1997) Am. J. Pathol., 150, 201-208; Pizer, E. S. e al. (1998) Cancer 83, 528-537). Therefore it is assumed that the growth of tumor cells with a high level of fatty acid synthesis could be suppressed by inhibition of fatty acid synthesis (Pizer, E. S. et al. (2000) Cancer Res., 60, 213-218; Kuhajda, F. P. et al. (2000) Proc. Natl. Acad. Sci. U.S.A., 97, 3450-3454). In WO 94/02108 it has been reported, that inhibition of fatty acid synthesis inhibits the growth of tumor cells implying ACL inhibitors as potential anti-tumor drugs. Similar observations have been made in U.S. Pat. No. 5,143,907 where the anti-tumor and anti-inflammatory effect of phosphite-borane compounds was thought to be correlated with the inhibition of cytoplasmatic synthesis of fatty acids and cholesterol.

In addition hypocitraturia of chronic metabolic acidosis is associated with an increase in ACL enzyme activity in renal cortical tissue and is partly reversed by inhibition of this enzyme (Melnick, J. Z. et al. (1996) J. Clin. Invest., 98, 2381-2387). These results suggest an important role of this enzyme in proximal tubular citrate metabolism and modulation of ACL enzyme activity may provide a target for treatment of hypocitraturia.

From the aforesaid it is evident, that ACL is a key enzyme in several biochemical pathways and that the modulation of ACL activity is of great importance for the treatment of a variety of diseases.

The object of the present invention is therefore to provide the use of modulators of ACL enzyme activity, new methods and medicaments for the modulation of ACL enzyme activity and the use of such compounds for the manufacture of medicaments for the treatment of pathophysiologic functions correlated with an increased or decreased ACL enzyme activity like hyperlipidaemia, hypercholesterolaemia, cardiovascular diseases, obesity, inflammatory diseases, tumors, diseases of the central nervous system, and hypocitraturia.

Other objects of the present invention are apparent for a skilled person on the basis of the following detailed description.

These objects are achieved on the basis of the unexpected finding that ACL is a substrate of the recently described protein human protein histidine phosphatase (hPHP) and its homologous variants and on the finding that hPHP modulates ACL activity by dephosphorylation of a phosphorylated histidine residue of ACL.

Accordingly, the present invention provides the use of a polypeptide with hPHP activity for the modulation of ACL enzyme activity.

Furthermore the present invention provides the use of compounds inhibiting hPHP activity like antibodies directed to hPHP or fragments thereof or a DNA sequence complementary to mRNA sequences encoding polypeptides with hPHP activity for the modulation of ACL enzyme activity.

The present invention furthermore provides the use of a compound with hPHP activity or a compound inhibiting hPHP activity for the manufacture of a medicament for the modulation of ACL enzyme activity.

The present invention provides also the use of polypeptides with hPHP activity or compounds inhibiting hPHP activity or a medicament comprising such compounds for the treatment of pathophysiologic conditions correlated to increased or decreased ACL enzyme activity like hyperlipidaemia, hypercholesterolaemia, cardiovascular diseases, obesity, inflammatory diseases, tumors, diseases of the central nervous system, and hypocitraturia

The present invention provides also methods for treating pathophysiologic conditions correlated with a increased or decreased ACL enzyme activity like hyperlipidaemia, hypercholesterolaemia, cardiovascular diseases, obesity, microbial infections, inflammatory diseases, tumors, diseases of the central nervous system and hypocitraturia comprising administering to patient a therapeutically effective amount of a polypeptide with hPHP activity or a compound with hPHP inhibiting activity or a medicament comprising such compounds.

Mammalian protein histidine phosphatase (hPHP) and its homologous variants are known from WO 00/52175 (Seq. No. 2-8). This protein has an apparent molecular weight of 14.000 and is N-terminally blocked.

Methods for the isolation, purification, characterization (p. 7, line 10 to p. 10, line 10) and the generation of antibodies (p. 7, line 13-30) are described in this application too.

Due to the fact that ACL is a substrate for the dephosphorylation activity of hPHP the phosphatase can be used for modulating the activity of ACL and therefore hPHP or a polypeptide having hPHP activity and pharmaceutical compositions comprising such a polypeptide can be used for the treatment of pathophysiologic functions correlated with an increased or decreased ACL enzyme activity like hyperlipidaemia, hypercholesterolaemia, cardiovascular diseases, obesity, inflammatory diseases, tumors, diseases of the central nervous system, and hypocitraturia.

The invention likewise includes the use of fragments, variants and mutants of hPHP, antibodies raised against these fragments, variants or mutants and DNA or RNA sequences complementary to the mRNA sequences of said fragments, variants or mutants for modulation ACL enzyme activity. Such fragments, variants and mutants of hPHP can be produced, for example, by random or controlled substitution, different splicing, deletion or addition of one or more nucleotides or amino acids, with the biologically activity being essentially retained

Thus, the present invention relates to the use of a polypeptide with hPHP activity for the modulation of ACL enzyme activity which comprises at least the amino acid sequence motif

DCECLGGGRISHQSQD

A further preferred polypeptide with hPHP activity for the modulation of ACL enzyme activity comprises at least the amino acid sequence motif

DCECLGGGRISHQSQDX¹KIHVYGYSMX²YGX³AQH

wherein X¹=K or R, X²=A or G and X³=P or R.

A further preferred polypeptide with hPHP activity for the modulation of ACL enzyme activity comprises at least the amino acid sequence motif

YHADIYDKVSGDMQKQGCDCECLGGGRISHQSQDKKIHVYGYSM.

All these partial sequences are highly conserved within the complete enzyme amino acid sequence and are deemed to be involved in the active site of said enzyme or have other biological or pharmaceutical relevance in mammals.

A especially preferred polypeptide with hPHP activity for the modulation of ACL enzyme activity comprises the amino acid sequence

(M)AVADLALIPDVDIDSDGVFKYVLIRVHSAPRSGAPAAESKEIVRGYK
WAEYHADIYDKVSGDMQKQGCDCECLGGGRISHQSQDKKIHVYGYSMAYG
PAQHAISTEKIKAKYPDYEVTWANDGY

The methionine residue at the N-terminal of the sequence is not obligatory.

It is a further object of the invention to provide the use of antibodies, preferably monoclonal humanized antibodies, raised against any one of the amino acid sequences described above for the inhibition of hPHP phosphatase activity and therefore for indirect modulation of ACL. Such antibodies can be generated using techniques well known to those of skill in the art.

Antibodies raised against hPHP, for example the antibody directed to the active site of hPHP having the amino acid sequence

CLGGGRISHQDK

(see p. 13, line 18, Seq. No. 10 of WO 00/52175) or an antibody directed to one of the amino acid sequences mentioned above can be used for the inhibition of hPHP phosphatase activity and therefore for indirect modulation of ACL.

Furthermore it is the object of the present invention to provide the use of DNA sequences or chemically modified DNA sequences complementary to the mRNA coding for the hPHP for inhibition of translation of hPHP and therefore for indirect modulation of ACL. Such a DNA sequence can easily be derived from the DNA sequence of hPHP described in WO 00/52175 in the sequence listing (Seq. No. 1) and may have one of the following sequences

| | |
|---|---|
| I) | TACCGCCACC GCCTGGAGCG AGAGTAAGGA CTACACCTGT |
| | AGCTGAGGCT GCCGCAGAAG TTCATACACG ACTAGGCTCA |
| | GGTGAGCCGA GGGGCGAGGC CCCGAGGCCG ACGTCTCTCG |
| | TTCCTCTAGC ACGCGCCGAT GTTCACCCGA CTCATGGTAC |
| | GCCTGTAGAT GCTGTTTCAC AGCCCGCTGT ACGTCTTCGT |
| | TCCGACGCTG ACACTCACAG ACCCGCCGCC CGCGTAGAGG |
| | GTGGTCTCAG TCCTGTTCTT CTAAGTGCAC ATGCCGATAA |
| | GGTACCGGAT ACCAGGACGG GTCGTGCGGT AAAGTTGACT |
| | CTTTTAGTTTC GGTTCATGGG GCTGATGCTC CAGTGGACCC |
| | GATTGCTGCC GATG |
| II) | CTGACACTCA CAGACCCGCC GCCCGCGTAG AGGGTGGTCT |
| | CAGTCCTG |
| III) | CTGACACTCA CAGACCCGCC GCCCGCGTAG AGGGTGGTCT |
| | CAGTCCTGTT CTTCTAAGTG CACATGCCGA TAAGGTACCG |
| | GATACCAGGA CGGGTCGTG |
| IV) | ATGGTACGCC TGTAGATGCT GTTTCACAGC CCGCTGTACG |
| | TCTTCGTTCC GACGCTGACA CTCACAGACC CGCCGCCCGC |
| | GTAGAGGGTG GTCTCAGTCC TGTTCTTCTA AGTGCACATG |
| | CCGATAAGGT AC |

Such DNA sequences can be generated using techniques well known to those of skill in the art.

The native as well as the recombinant polypeptide(s), antibodies or DNA sequences mentioned above can be applied to patients suffering from pathophysiologic functions correlated with an increased or decreased ACL enzyme activity like hyperlipidaemia, hypercholesterolaemia, cardiovascular diseases, obesity, microbial infections, inflammatory diseases, tumors, diseases of the central nervous system, and hypocitraturia directly or within pharmaceutical compositions comprising said compounds and a pharmaceutically acceptable diluent, carrier or excipient therefor.

As used herein, the term “pharmaceutically acceptable carrier” means an inert, non toxic solid or liquid filler, diluent or encapsulating material, not reacting adversely with the active compound or with the patient. Suitable, preferably liquid carriers are well known in the art such as sterile water, saline, aqueous dextrose, sugar solutions, ethanol, glycols and oils, including those of petroleum, animal, vegetable, or synthetic origin, for example, peanut oil, soybean oil and mineral oil.

The formulations according to the invention may be administered as unit doses containing conventional non-toxic pharmaceutically acceptable carriers, diluents, adjuvants and vehicles which are typical for parenteral administration.

The term “parenteral” includes herein subcutaneous, intravenous, intra-articular and intratracheal injection and infusion techniques. Also other administrations such as oral administration and topical application are suitable. Parenteral compositions and combinations are most preferably administered intravenously either in a bolus form or as a constant fusion according to known procedures.

When the compounds of the invention are formulated as a tablet capsule or powder, usual carriers and excipients such as magnesium carbonate, calcium carbonate, sodium bicarbonate, magnesium stearate, calcium stearate, talc, lactose, microcrystalline cellulose, methyl cellulose, sodium carboxymethyl cellulose starch and anhydrous silica, lubricants such as hydrated castor oil, magnesium stearate, sodium lauryl sulfate and sugar, pectin, dextrin, tragacanth, a low-melting wax, cocoa butter, alginates, gelatin, polyvinyl pyrrolidone, polyethyl glycols, quaternary ammonium compounds and the like as well as binders such as starch, glucose, gum arabicum and mannitol can be used. The tablets or capsules may be coated according to methods well known in the art.

Oral liquid preparations may be in the form of aqueous or oily suspensions, solutions, emulsions, syrups or elixirs, or may be presented as a dry product for reconstitution with water or another suitable vehicle before use. Such liquid preparations may contain conventional additives like suspending agents, emulsifying agents, non-aqueous vehicles and preservatives.

Topical applications may be in the form of aqueous or oily suspensions, solutions, emulsions, jellies or preferably emulsion ointments.

Unit doses according to the invention may contain daily required amounts of the compound according to the invention, or sub-multiples thereof to make up the desired dose. The optimum therapeutically acceptable dosage and dose rate for a given patient (mammals, including humans) depends on a variety of factors, such as the activity of the specific active compound employed, the age, body weight, general health, sex, diet, time and route of administration, rate of clearance, enzyme activity (units/mg protein), the object of the treatment, i.e., therapy or prophylaxis and the nature of the disease to be treated which are known to the skilled person.

Therefore, in compositions and combinations in a treated patient (in vivo) a pharmaceutical effective daily dose of the active compound of this invention is between about 0.01 and 100 mg/kg body weight, preferably between 0.1 and 10 mg/kg body weight. According to the application form one single dose may contain between 0.01 and 10 mg of the active compound.

The modulators of ACL enzyme activity of this invention may be used to treat patients suffering from cancers which have an elevated level of fatty acid synthesis or depend on endogenous fatty acid. Characteristic carcinomas amenable to treatment include those of bladder, salivary gland, skin adnexae, bile duct, endocervix, ectocervix, and vagina, esophagus, nasopharynx and oropharynx, or those of germ cell origin, and mesothelioma. In particular, carcinomas or adenocarcinomas of the stomach, endometrium, kidney, liver and lung, as well as melanoma are treatable according to this invention. Breast, colon and rectum, prostate, and ovary, are especially suitable types of adenocarcinomas for the application of this therapy.

Endogenous fatty acid synthesis by such cells will preferably occur at a rate of incorporation greater than 10 fmoles of acetyl-CoA into acyl glyceride per 200,000 cells per minute. Preferred patients may be identified because they have tumors containing cells which express ACL or other enzymes of the fatty acid synthesis pathway, such as acetyl CoA carboxylase (ACC), at levels higher than the level found in the surrounding normal (e.g., non-neoplastic) tissue. Such cells are aggressive tumor cells and result in decreased survival, increased metastasis, increased rates of clinical recurrence and overall worsened prognosis. Since many tumor cells are extremely dependent on endogenous fatty acid synthesis, lower activity levels of fatty acid synthesis need not exclude a specific tumor as a candidate for therapy with the active compounds of the present invention. Fatty acid synthesis would be reduced or stopped by inhibitors of ACL. The result would be deprivation of membrane lipids, which would cause cell death. Normal cells, however, would survive as they are able to import circulating lipid.

The presence of ACL in cells of the carcinoma may be detected by any suitable method, including activity assays or stains, immunoassays using anti-ACL antibodies, assays measuring ACL mRNA, and the like.

Expression of ACL may be determined directly in tumor tissue samples obtained through procedures such as biopsies, resections or needle aspirates, using assays such as immunohistochemistry, cytosol enzyme immunoassay or radioimmunoassay, in situ hybridisation of nucleic acid probes with mRNA targets having ACL sequences, or direct measurement of enzyme activity. Expression of ACL by the tumor may be indirectly measured in biological fluid samples obtained from patients, such as blood, urine, serum, lymph, saliva, semen, ascites, or especially plasma, using any suitable assays.

Cells that require endogenously synthesized fatty acid are widespread among carcinomas, particularly the most virulent carcinomas. While it is preferred that the presence of ACL be determined prior to treatment, the skilled clinician will recognize that such determination is not always necessary. Treatment of a carcinoma patient with an inhibitor of ACL, which results in reduction of tumor burden demonstrates the presence of ACL in the tumor. Such empirical treatment of carcinomas is also within the contemplation of this invention.

The modulators of ACL enzyme activity of the present invention are also useful in conjunction with other chemotherapeutic agents. Since no presently prescribed cancer chemotherapeutic agents are specifically active against the fatty acid synthase pathway, the use of the compounds of the present invention will complement existing anti-cancer drugs, particularly antimetabolic drugs that target other anabolic or catabolic pathways.

Chemotherapeutic agents which may be used in conjunction with the compounds of the present invention includes, according to this invention, agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Chemotherapeutic agents according to the invention are preferably natural or is synthetic chemical compounds, but biological molecules, such as proteins, antibodies, chemokines, cytokines, polypeptides etc. are not excluded. There are large numbers of chemotherapeutic agents available in commercial use, in clinical evaluation and in pre-clinical development, which could be included in the present invention.

Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11,10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

The modulators of ACL enzyme activity of this invention may furthermore be used to treat patients suffering from hypercholesterolaemia and/or hyperlipidaemia and preventing the development of consequent disorders like atherosclerosis and pancreatitis, as well as treatment of metabolic disorders like obesity.

It is now widely accepted that treatment of even moderate type II hypercholesterolaemia results in a reduction in mortality and morbidity due to coronary heart disease.

Increased plasma concentrations of low density lipoprotein, the hallmark of type II hypercholesterolaemia are due to a variety of genetic and environmental factors resulting in increased LDL synthesis, decreased LDL catabolism or combinations of both. Current therapies for treatment of hypercholesterolaemia are directed towards stimulation of LDL catabolism (bile acid sequestrants and HMG-CoA reductase inhibitors) as well as inhibition of LDL synthesis (nicotinic acid and maxepa fish oil).

The compounds of the present invention act by modulation of the ACL enzyme activity, so inhibiting cholesterol synthesis and fatty acid synthesis resulting in lowered plasma cholesterol and triglyceride levels. The present invention therefore provides the use of inhibitors of ACL enzyme activity for use in therapy, in particular for lowering serum triglyceride and cholesterol levels in the treatment of mixed hyperlipidaemia (type (IIb)). In addition, the use of the compounds of the present invention is expected to exhibit a beneficial effect in preventing the development of consequent disorders like atherosclerosis and pancreatitis as well as the treatment of metabolic disorders like obesity.

Furthermore, the compounds of the present invention may be used for promoting fat loss from the stimulation of fat oxidation because with inhibition of ACL enzyme activity little acetyl CoA reaches cytoplasm. This limits the availability of malonyl-CoA which acts as inhibitor for carnitine acyltransferase, a enzyme necessary for the fat burning process in mitochondria. With a low level of malonyl CoA the degradation of fatty acid is induced and consequently the compounds of the present invention can promote fat loss. This effect is supported by the fact, that activation of fatty acid oxidation in the liver also tends to stimulate gluconeogenesis which in turn may replenish the stores of liver glycogen and send a message of satiety to the brain centre. Therefore, the present invention provides the use of inhibitors of ACL enzyme activity for promoting fat loss and as appetite suppressants.

In addition the present invention provides the use of the compounds of the present invention for the treatment of neurodegenerative diseases.

For example, Alzheimer's disease is a genetically heterogeneous group of progressively fatal neurological diseases characterized pathologically by accumulation of amyloid plaques in brain and clinically by impairment of recent memory leading to dementia and death. In addition to the cases of Alzheimer's disease linked to genetic causes, sporadic cases, without an apparent family history of the disease, also occur. For example pathological changes characteristic of Alzheimer's disease occur after head trauma or after inflammatory diseases stimulating production of the cytokine interleukin-1. The early symptom of the disease is loss of recent memory associated with impairment and death of cell in the hippocampus accounting for the early impairment of recent memory. Measurement of the hippocampal volumes using magnetic resonance imaging shows that atrophy of hippocampus occurs prior to the clinical onset of memory loss and progresses with a loss of volume of about 8% per year during the 2 years over which symptoms first appeared.

The diagnosis of Alzheimer's disease is made clinically by this impairment in recent memory, associated with lesions in the hippocampal portion of the temporal lobe.

While Alzheimer's disease of the familial or the sporadic type is the major dementia found in the aging population, other types of dementia are also found. These include but are not limited to: the fronto-temporal degeneration associated with Pick's disease, vascular dementia, senile dementia of Lewy body type, dementia of Parkinsonism with frontal atrophy, progressive supranuclear palsy and corticobasal degeneration and Downs syndrome associated Alzheimers'. Plaque formation is also seen in the spongiform encephalopathies such as CJD, scrapie and BSE.

In addition to amyloid plaques, decreased brain acetyl choline levels is a further pathological characteristic of Alzheimer's disease. Modest clinical improvement in symptoms can occur by treatment with acetyl choline esterase inhibitors presumably by increasing cholinergic efferents originating in the septal nuclei and traversing Broca's diagonal band to hippocampus in the anterior portion of the Limbic system of brain.

The same effect might be achieved by stimulation of ACL enzyme activity, because the acetyl-CoA necessary for formation of acetyl choline derives from the enzymatic conversion of citrate to acetyl-CoA and oxaloacetate.

Due to the observations that the anti-inflammatory effect of phosphite-borane compounds was thought to be correlated with the inhibition of cytoplasmatic synthesis of fatty acids and cholesterol (U.S. Pat. No. 5,143,907) the present invention in addition provides the use of the compounds of the present invention for the treatment inflammatory diseases

Nonlimiting examples of inflammatory disease according to the present invention are acute glomerulonephritis, acute synovitis adult respiratory distress syndrome, atherosclerosis, autoimmune thyroiditis, autoimmune hemolytic anemias, bronchitis, cachexia, conjunctivitis, dermatosis with acute inflammatory components, gouty arthritis, graft vs. host reactions, Grave's disease, Hashimoto's thyroiditis, hemodialysis, inflammatory bowel disease including Crohn's disease and ulcerative colitis, insulin-dependent diabetes mellitus, leukapheresis, multiple sclerosis, myasthenia gravis, necrotizing enterocolitis, organ/tissue transplant rejection, osteoarthritis, dermatitis, psoriatic arthritis, psoriasis, Raynaud's syndrome, reactive arthritis, Reiter's syndrome, rheumatic fever, rheumatoid arthritis, rhinitis, rubella arthritis, systemic lupus erythematosus, traumatic arthritis, vasculitis and uveitis.

Furthermore, the compounds of the present invention may be used for the treatment of hypocitraturia, because it was shown that chronic metabolic acidosis increases the activity and protein abundance of renal cortical ACL and that upregulation of this enzyme plays an important role in the generation of hypocitraturia. Therefore the compounds of the present invention may be used for the treatment of hypocitraturia.

### SHORT DESCRIPTION OF THE FIGURES

FIG. 1: hPHP dependent dephosphorylation of the substrate ACL; dephosphorylation without and with hPHP; lane 1: control (without hPHP), 2: 280 ng hPHP, 3: 210 g hPHP, 4: 140 ng hPHP, 5: 70 ng hPHP, 6: 28 ng hPHP. The protein with an apparent molecular weight of about 120 kDa was dephosphorylated in a hPHP-concentration dependent manner.

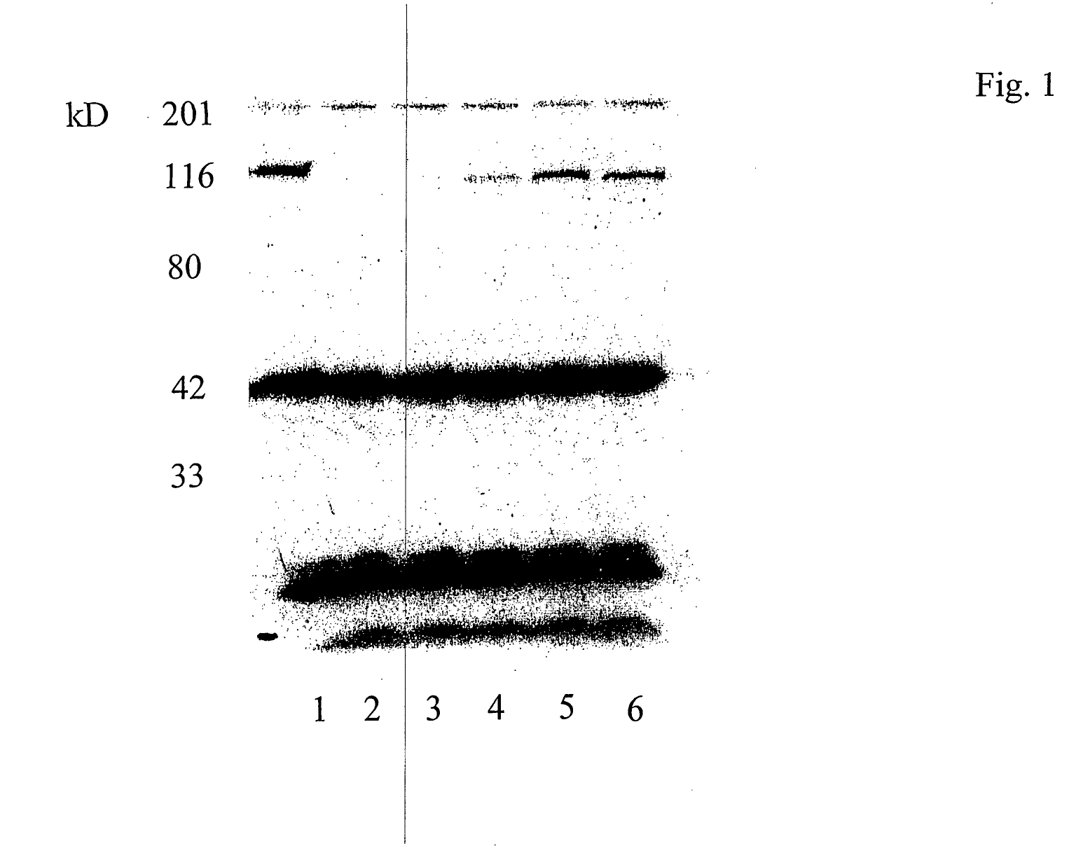

FIG. 2: Identification of ACL; left panel: overlay after autoradiography treatment (grey spot) and after. Coomassie stained gel electrophoresis; 1: molecular markers, 2: rat liver soluble extract, 3: partially purified ACL; right panel: Coomassie stained gel electrophoresis; 1: molecular markers, 2: rat liver soluble extract. ACL is indicated by the arrow.

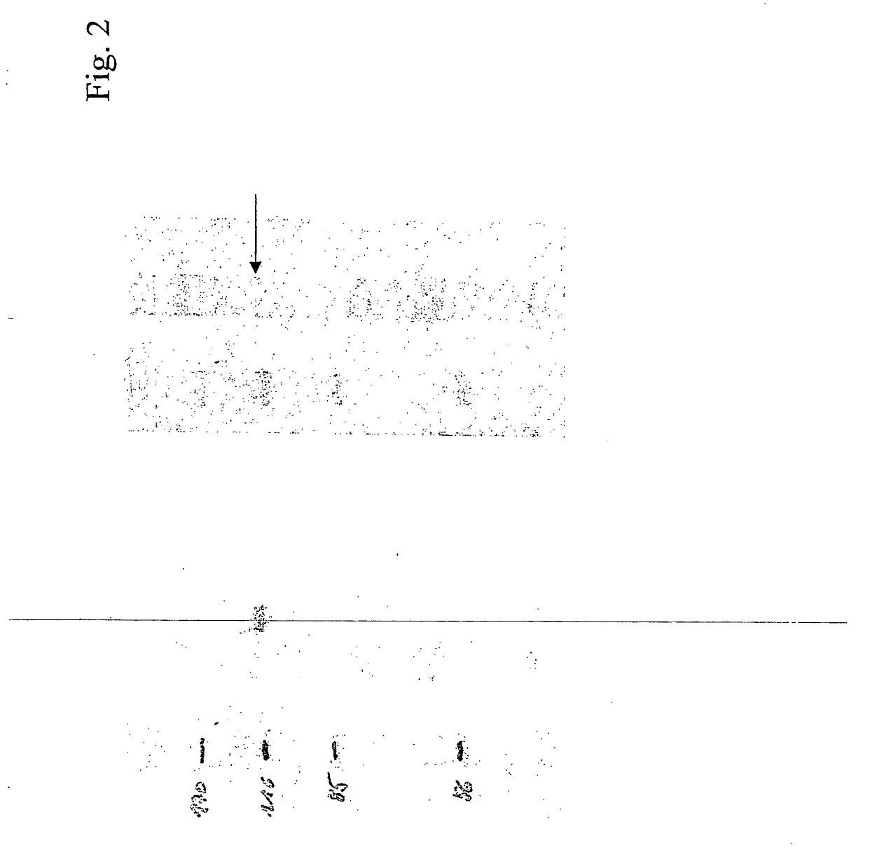

### EXAMPLES

### Example 1

Substrate Determination of hPHP

To determine a vertebrate substrate for hPHP1 a ³²P-labelled rabbit liver extract was screened. Rabbit liver extracts were labelled according to published protocols to selectively obtain proteins phosphorylated on histidine residues (FEBS Lett 1995;364, 63-3). This was verified by acid and alkaline treatment of the proteins blotted onto PVDF membranes and subsequent autoradiography. Addition of hPHP selectively resulted in dephosphorylation of a protein with mobility on SDS-gels of 110K. The hPHP substrate protein was isolated and subsequently identified as ATP-citrate lyase (ACL). ACL is known to autophosphorylate at histidine 764 in the course of catalysis.

### Example 2

PHP Assays

Phosphorylation of the phosphatase substrate cheA was prepared. Unincorporated [γ-³²P]ATP was removed using a Sephadex G-50 column. hPHP was incubated for 30 min at 37° C. in a 40 μl reaction mixture containing 0.6 ng [³²P]cheA (0.21 pmol [³²P]/ml), 25 mM TEA pH 7.5, 10 mM MgCl₂, and 0.1% β-mercaptoethanol. Assays were stopped by adding 10 μl 0.5 M EDTA and 150 μl methanol/acetone (1:1), centrifuged at 15,000 g for 5 min, and the supernatant analysed for [³²P] content. PHP was diluted so that phosphate release was kept within the linear range (<25%).

Phosphorylation of a rabbit liver soluble extract including the 110K phosphatase substrate was prepared as described. The 15 μl dephosphorylation reactions contained 5-50 ng PHP, 25 mM TEA pH 7.5, 0.1% β-mercaptoethanol and 60 μg of the phosphorylated extract. Assays were stopped after 30 min at 37° C. by addition of sample buffer. Reaction products were analysed on 10% SDS-PAGE followed by autoradiography.

### Example 3

Purification of hPHP and its Substrate

The soluble extract from rabbit liver was used as starting material. Buffer A consisted of 20 mM TEA, 1 mM EDTA, 0.1% β-mercaptoethanol, 0.02% NaN₃, pH 7.5 supplemented with NaCl or Mg²⁺, and was used for all purification steps except during Blue Sepharose 6 Fast Flow, when 0.1 mM EDTA was present (buffer B).

The extract was loaded on SOURCE 30Q and eluted with buffer A plus 0.2 M NaCl. Fractions containing hPHP activity were concentrated by 90% (NH₄)₂SO₄ followed by chromatography on HiLoad 26/60 Superdex 75 run in buffer A. The elution volume of 11-21K was pooled, adjusted to 10 mM Mg²⁺ and applied to Blue Sepharose equilibrated in buffer B containing 10 mM Mg²⁺. hPHP eluted in buffer B supplemented with 0.2 M NaCl.

ACL was partially purified from the soluble extract from rabbit liver by Source 30Q, HiLoad 26/60 Superdex 200 and MonoQ as described (Hoffmann, G. E. et al. (1979) Hoppe-Seyler's Z. Physiol. Chem. 360, 1445-51).

### Example 4

Anti-Histidine Phosphatase Antibodies

Anti-Histidine phosphatase antibodies were generated against different regions of the protein. The peptides were synthesized using standard FMOC-chemistry. For immunization the peptides were injected (4 injections) each into two rabbits and four blood samples were taken. Final bleeding was taken after ca. 3 month.

The generated antibodies are usefull for detection and localization of the histidine phosphatase.

Furthermore, the different regions within the molecule can be analyzed individually. Especially the highly conserved central part of the histidine phosphatase containing the following amino acid sequence:

DCECLGGGRISHQSQD

is assumed to contain the active site responsible for the proteins function in vivo. The anti-peptide antibody against this region is for inhibitory or neutralizing use.

1. Use of a polypeptide having the biological activity of a Protein Histidine Phosphatase (PHP) which has a high specificity for phosphohistidine or a homologue variant for the modulation of ATP-citrate-lyase (EC 4.1.3.8) activity.

2. Use of a polypeptide according to claim 1, having a molecular weight of 13.000-15.000,

3. Use of a polypeptide according to claim 1, whereas the polypeptide comprises at least the amino acid sequence motif selected from the group of

| | |
|---|---|
| I) | DCECLGGGRISHQSQDX“KIHVYGYSMX²YGX³AQH |
| | wherein X¹ = K or R, X² = A or G and X³ = PorRor |
| II) | DCECLGGGRISHQSQD |
| or | |
| III) | (M)AVADLALIPDVDIDSDGVFKYVLIRVHSAPRSGAPAAESKE |
| | IVRGYKWAEYHADIYDKVSGDMQKQGCDCECLGGGRISHQSQDK |
| | KIHVYGYSMAYGPAQH AISTEKIKAKYPDYEVTWANDGY. |

4. Use of an antibody or a fragment thereof directed to a polypeptide having a protein histidine phosphatase activity according to claim 1 for the modulation of ATP-citrate-lyase (EC 4.1.3.8) activity.

5. A DNA sequence complementary to the mRNA sequence of protein histidine phosphatase having at least one of the following sequences

| | |
|---|---|
| I) | TACCGCCACC GCCTGGAGCG AGAGTAAGGA CTACACCTGT |
| | AGCTGAGGCT GCCGCAGAAG TTCATACACG ACTAGGCTCA |
| | GGTGAGCCGA GGGGCGAGGC CCCGAGGCCG ACGTCTCTCG |
| | TTCCTCTAGC ACGCGCCGAT GTTCACCCGA CTCATGGTAC |
| | GCCTGTAGAT GCTGTTTCAC AGCCCGCTGT ACGTGTTCGT |
| | TCCGACGCTG ACACTCACAG ACCCGCCGCC CGCGTAGAGG |
| | GTGGTCTCAG |
| | TCCTGTTCTT CTAAGTGCAC ATGCCGATAA GGTACCGGAT |
| | ACCAGGACGG GTCGTGCGGT AAAGTTGACT CTTTTAGTTTC |
| | GGTTCATGGG GCTGATGCTC CAGTGGACCC GATTGCTGCC |
| | GATG |
| II) | CTGACACTCA CAGACCCGCC GCCCGCGTAG AGGGTGGTCT |
| | CAGTCCTG |
| Ill) | CTGACACTCA CAGACCCGCC GCCCGCGTAG AGGGTGGTCT |
| | CAGTCCTGTT CTTCTAAGTG CACATGCCGA TAAGGTACCG |
| | GATACCAGGA CGGGTCGTG |
| IV) | ATGGTACGCC TGTAGATGCT GTTTCACAGC CCGCTGTACG |
| | TCTTCGTTCC GACGCTGACA CTCACAGACC CGCCGCCCGC |
| | GTAGAGGGTG GTCTCAGTCC TGTTCTTCTA AGTGCACATG |
| | CCGATAAGGT AC |

6. Use of a DNA sequence according to claim 5 for the inhibition of the translation of protein histidine phosphatase, for the modulation of ATP-citratelyase (EC 4.1.3.8) activity.

7. Use of a compound according to claim 1 for the manufacture of a medicament for the treatment of pathophysiologic conditions susceptible to the modulation of ATP-citrate-lyase (EC 4.1.3.8) activity.

8. Use of a compound according to claim 7, wherein the pathophysiologic condition is selected from the group of hyperlipidaemia, hypercholesterolaemia, cardiovascular diseases, obesity, inflammatory diseases, tumors, diseases of the central nervous system, and hypocitraturia.

9. Use of a compound according to claim 1 for the manufacture of a medicament for controlling weight, promoting fat loss and for appetite suppression.
